(19) United States
(12) Patent Application Publication
(10) Pub. No.: US 2007/0134781 A1
(43) Pub. Date: Jun. 14, 2007

(54) Method for producing bioethanol from a lignocellulosicbiomass and recycled paper sludge

(76) Inventor: Foster Aryi Agblevor, Christiansburg, VA (US)

Correspondence Address: GREENBERG TRAURIG, LLP, MET LIFE BUILDING, 200 PARK AVENUE, NEW YORK, NY, 10166, US

(21) Appl. No.: 11/301,970

(22) Filed: Dec. 12, 2005

(51) Int. Cl. C12P 7/14; C12P 7/06

(52) U.S. Cl. 435/161; 435/162

(57) ABSTRACT

A method producing ethanol by combining an plant fiber material containing calcium carbonate with at least one lignocellulosic agricultural residue into a mixture. The plant fiber material can be a paper sludge. The mixture is then hydrolyzed and the resultant hydrolysate is then fermented into ethanol.

### BACKGROUND OF THE INVENTION

The production of ethanol for fuel applications is becoming increasingly important in the world. In the United States, current ethanol demand for fuel applications is estimated at 2.5 billion gallons per annum, and this is expected to increase to 4.5 billion gallons per annum by 2005 when methyl tertiary butyl ether (MTBE) is phased out of gasoline in California and other states. Ethanol is currently produced from the fermentation of cornstarch. Corn-ethanol is not energy efficient non-economically competitive, as testified to by the fact that the product has to be subsidized at $0.53 per gallon. Further, Title IX of the 2002 Farm Bill and current USA Department of Energy and USA Department of Agriculture efforts are targeted at producing inexpensive ethanol from biomass resources. The goal is to promote a cleaner environment and reduce dependence on imported petroleum products.

The major challenges in converting lignocellulosic biomass to ethanol include high cost of dedicated biomass feedstock, pretreatment of lignocellulosic feedstock to release sugars for fermentation, poor fermentation of pentose sugars to ethanol by wild type microorganisms, and toxicity of biomass hydrolysates to both recombinant and wild type fermentative microorganisms.

The biomass hydrolyzates are usually detoxified by methods such as evaporation, solvent extraction, overliming, neutralization, ion-exchange, enzyme degradation or a combination of several of these methods. Prior to the present invention, the most effective known method has been the overliming process in which lime (calcium hydroxide or calcium carbonate) is reacted with and precipitates chemical components toxic to both microbial growth and ethanol fermentation and inhibits cellulase enzyme.

What is needed is a more economical and efficient method for producing ethanol.

### SUMMARY OF THE INVENTION

The current invention addresses a major problem in converting biomass to ethanol by exploiting the unique properties of the biomass feedstocks to solve the problem of inefficient ethanol production. Ethanol production from lignocellulosic biomass feedstocks is less intensive and will be economically competitive.

In one embodiment, the invention provides a method of producing ethanol. This method includes a mixture that is made by combining a plant fiber material containing calcium carbonate with at least one lignocellulosic agricultural residue. The agricultural residue is treated to increase accessibility of biopolymers to hydrolytic agents, and may be treated either alone or in combination with the plant fiber material. Then the mixture can be hydrolyzed. The resultant hydrolysate is then placed in the presence of organisms which can convert cellulosic material into ethanol, thereby fermenting the mixture to form ethanol.

In particular, the process may be comprised by using a paper sludge as the plant fiber material. Cotton gin waste (CGW) may be employed as the lignocellulosic agricultural residue. The lignocellulosic agricultural residue can also include, inter alia, corn stover, rice hull, or sugar cane bagasse.

Treatment of the agricultural residue may be by steam pretreatment. The plant fiber material and lignocellulosic agricultural residue may be mixed in specific proportions before the steam pretreatment. For example the ratio of lignocellulosic agricultural residue to plant fiber material can be a ratio of 1:1, 2:1, 3:1, 4:1, or 5:1. In the alternative, the lignocellulosic agricultural residue may be steam pretreated separately and then mixed with appropriate proportions of plant fiber material. The presence of the calcium carbonate, and titanium dioxide in the plant fiber material renders these mixtures readily hydrolyzed and fermented to ethanol by fermentative microorganisms. Fermentation is efficient and ethanol yields are high.

In another embodiment, the invention provides a method of producing ethanol comprising mixing a plant fiber material that contains calcium carbonate and titanium dioxide with at least one lignocellulosic agricultural residue into a mixture such that mixture is fermentably detoxified for ethanol production by the addition of the plant fiber material alone. The method then allows the mixture to be processed into ethanol.

### DETAILED DESCRIPTION OF THE INVENTION

Ethanol production from lignocellulosic biomass is currently researched in several laboratories in the country. The major challenges in converting lignocellulosic biomass to ethanol include high cost of dedicated biomass feedstock, pretreatment of lignocellulosic feedstock to release sugars for fermentation, poor fermentation of pentose sugars to ethanol by wild type microorganisms, and toxicity of biomass hydrolysates to both recombinant and wild type fermentative microorganisms.

The biomass hydrolyzates are usually detoxified by methods such as evaporation, solvent extraction, overliming, neutralization, ion-exchange, enzyme degradation or a combination of several of these methods. The most effective method to date is the overlining process in which lime (calcium hydroxide or calcium carbonate) is reacted with and precipitates chemical components toxic to both microbial growth and ethanol fermentation.

The present invention provides a method for producing a biomass to ethanol. This method includes mixing a plant fiber material containing calcium carbonate and titanium dioxide with at least one lignocellulosic agricultural residue. The plant fiber material can be a paper sludge. The lignocellulosic agricultural residue is treated to increase the accessibility of biopolymers to hydrolytic agents. It may be treated either alone or in combination with the agricultural residue. Then the mixture is hydrolyzed and the resultant hydrolysate is fermented into ethanol.

Recycled Paper Sludge

Since the plant fiber material used in the invention can be a paper sludge, description of one form of paper sludge, Recycled Paper Sludge, is provided below for a better understanding of the present invention. That said, a suitable plant fiber material includes anything fine paper that contains calcium carbonate and titanium dioxide. Examples include: (a) fine paper sludge, (b) coated paper sludge, (c) coated fine paper sludge, (d) groundwood paper sludge, (e) recycle mixed office paper sludge, (f) recycled newsprint, (g) de-inked pulp mill sludge, (h) de-inked paper mill sludge, (i) old corrugated containers. Recycled paper sludge (RPS) is the residue produced after the recycling of paper and dewatering of the residue. This residue may contain as much as from about 50% to about 60% water. These residues contain from about 50% to about 70% low quality cellulosic fibers and paper additives (from about 30% to about 50%) that cannot be used for paper making and are therefore disposed in landfills. About 4.5 million tons of these materials are produced annually from paper recycling operations in the United States of America. A description of RPS can be found in U. S. Pat. No. 5,777,086 to Klyosov, et al., incorporated in its entirety herein.

About 90% of the organic fraction of the fiber is cellulose and the rest is lignin and hemicellulose. The RPS also contains inorganic compounds (ash) added to the pulp to improve its paper making properties. The ash content of RPS ranges from about 5% to about 30% by mass of the sludge. A major component of the RPS ash is calcium carbonate that is added to the pulp during paper making. The calcium carbonate has similar properties as the lime used for the overliming detoxification of the biomass hydrolysates before fermentation. It also contains titanium dioxide in small quantities that has useful properties for detoxification.

RPS can be hydrolysed as is or steam pretreated before hydrolysis to increase the efficiency of the process. The hydrolysate contains mostly glucose that can be easily fermented to ethanol by fermentative microorganisms.

Cotton Gin Waste

A lignocellulosic agricultural residue is mixed with the plant fiber material to make a mixture that can be processed to produce ethanol. The lignocellulosic agricultural residue that may be used includes, inter alia, cotton gin waste (CGW). A description of CGW is provided for a better understanding of the present invention. CGW is the residue from the ginning of raw cotton to produce cotton fibers. CGW consists of cotton fibers, immature boles, sticks and grass, cottonseed, hulls, and inorganic material (ash). The total carbohydrate fraction of this material is from about 40% to about 55%. About 75% of the total carbohydrate is cellulose because of the significant fraction of cotton fiber in the feedstock.

Unlike the RPS, the non-cotton fiber fraction of the CGW cannot be readily hydrolysed and fermented into ethanol. This feedstock requires a pretreatment to increase accessibility of the biopolymers to hydrolytic agents. During steam pretreatment, there is a loss of carbohydrates. Most of the xylan, mannan, galactan, arabinan and a small fraction of the cellulose can be lost from the residual fiber. These lost fractions may be either in the aqueous fraction as monomeric, oligomeric, or thermochemical decomposition products. Thermochemical decomposition compounds such as furfural, hydroxymethylflurfural, and acetic acid can be in the steam treated material.

The thermochemical decomposition products of CGW are toxic to fermentative microorganisms, which inhibit both cell growth and fermentation of the hydrolysates to ethanol by an organism capable of converting cellulosic material to ethanol such as recombinant Escherichia coli KO11.

The current invention addresses some of the above-mentioned obstacles by exploiting the unique properties of the biomass feedstocks to solve the problem. An exemplary process consists of using mixtures of RPS and a lignocellulosic agricultural residue such as CGW to produce ethanol. The RPS and CGW are mixed in specific proportions before steam pretreatment. For example, the RPS and CGW may be mixed such that the proportion of lignocellulose to plant fiber material is at a ratio of 1:1, 2:1, 3:1, 4:1, or 5:1. The CGW can also steam pretreated separately and then mixed with appropriate proportions of RPS. These mixtures are easily hydrolyzed and fermented to ethanol by fermentative microorganisms without any of the above detoxification processes because the hydrolysates are detoxified by the processing conditions. Fermentation is efficient and ethanol yields are high.

The process exploits the fact that the RPS contains calcium carbonate, which is the same detoxifying agent used in the overliming processes. Additionally, the presence of titanium dioxide can also act as a detoxifying agent by binding to the toxicants. During steam pretreatment, the RPS is defibrated and the fine calcium carbonate fraction is exposed. In the steam explosion case, the defibration is achieved during the explosion process. In another example, in the case where the RPS is slurried in a blender, defibration is achieved by this method. The calcium carbonate particles react with the steam explosion-induced degradation products such as acetic acid and lignin decomposition products and precipitate them into the mixture of steam condensate, fiber, and oligomeric products. The steam treated product is therefore non-toxic to microorganisms.

The RPS can be combined with other lignocellulosic agricultural residues such as corn stover, rice hull, sugar cane bagasse and others. The ratio of feedstock to RPS is expected to change with various feedstocks. The RPS ratio will be adjusted according to each feedstock for effective detoxification. For example, in feedstocks such as aspen wood, oat hulls, cotton gin waste, corn stover, or sugar cane bagasse the ratio of 1:1 yields good results in terms of enzyme hydrolysis, sugar yield, and fermentation. Ratios of 1:1, 2:1, 3:1, 4:1, or 5:1 may also be used.

The following examples further illustrate details for the process of the present invention, and the preparation of the compositions of this invention. The invention, which is set forth in the foregoing disclosure, is not to be limited either in spirit or scope by these examples. Those skilled in the art will readily understand that known variations of the conditions and processes of the following preparative procedures can be used to prepare these substances.

### EXAMPLES

### Example 1

In the first example, RPS containing about 50% or more moisture was mechanically mixed with the CGW. This mixture is loaded into a batch steam explosion gun and saturated steam is admitted into the reaction chamber until the feed temperature is about 200 degrees C. The steam source is then closed and the reaction is allowed to proceed for from about 2 minutes to about 5 minutes. The steam valve at the bottom of the reactor is then opened and the biomass is explosively decompressed into a cyclone separator. The cyclone separates the steam and other gaseous components from the fiber fraction.

The fiber fraction was slurried to about 20% solids and hydrolyzed with cellulase enzyme at about 50° C., pH 5 for about 72 hours. The hydrolysate was fermented with E. coli KO11 to ethanol. In the absence of RPS the highest ethanol yield was about 50 gallons per ton of CGW. However, with the inclusion of the RPS, the glucose concentration in the mixture indicates that the ethanol yield increases considerably, with results of up to 90% of the ethanol yield being achieved.

### Example 2

In another example, RPS is first slurried with water and defibrated. The slurry is then added to a steam-treated CGW to a concentration of 20% solids. The degradation products from the treated CGW react with the calcium carbonate contained in the RPS and is precipitated. The slurry is hydrolyzed with a cellulase enzyme preparation at pH 5, at about 50 degrees C. for about 72 hours in shake flasks. The hydrolysate is then fermented with E. coli KO11 to ethanol.

### Example 3

In yet another example RPS containing over 50% water is mechanically mixed with ground corn stover and steam treated. The recovered fiber is hydrolysed with cellulase enzyme preparation for about 72 hours. The hydrolysate is fermented with E. coli KO11 to ethanol.

It should be understood that the above description is only representative of illustrative embodiments and examples. For the convenience of the reader, the above description has focused on a limited number of representative examples of all possible embodiments, examples that teach the principles of the invention. The description has not attempted to exhaustively enumerate all possible variations or even combinations of those variations described. That alternate embodiments may not have been presented for a specific portion of the invention, or that further undescribed alternate embodiments may be available for a portion, is not to be considered a disclaimer of those alternate embodiments. One of ordinary skill will appreciate that many of those undescribed embodiments, involve differences in technology and materials rather than differences in the application of the principles of the invention. Accordingly, the invention is not intended to be limited to less than the scope set forth in the following claims and equivalents.

1. A method of producing ethanol comprising:
a) treating at least one lignocellulosic agricultural residue to increase accessibility of biopolymers to hydrolytic agents;
b) mixing a plant fiber material comprising calcium carbonate with the at least one lignocellulosic agricultural residue;
c) hydrolyzing the mixture of b) into a hydrolysate; and
d) fermenting the hydrolysate of c) in the presence of an organism capable of converting cellulosic material to ethanol and forming ethanol.

2. The method of claim 1 wherein the at least one lignocellulosic agricultural residue further comprises:
a cotton gin waste;
a corn stover;
a rice hull; or
a sugar cane bagasse.

3. The method of claim 1 wherein the plant fiber material comprises a paper sludge.

4. The method of claim 3 wherein the paper sludge comprises:
an ash content ranging from about 5% to about 30% by mass of the paper sludge, said ash content including the calcium carbonate at a minimum of about 5%.

5. The method of claim 1 wherein the least one lignocellulosic agricultural residue is mixed with the plant fiber material prior to treatment.

6. The method of claim 1 wherein said plant fiber material is defibrated prior to treatment.

7. The method of claim 5 wherein the method further comprises:
the treating of a) comprising:
i) admitting into a batch steam explosion reactor saturated steam from a steam source until the mixture of b) is about 200 degrees Celsius;
ii) reacting said mixture for from about two to about five minutes;
iii) decompressing said mixture into a separator;
iv) separating out a gaseous component of said mixture; and
v) slurrying said mixture to about 20% solids; and
and wherein said hydrolyzing of c) is at about 50 degrees Celsius, ph 5 for about 72 hours;
and said organism in the fermenting of d) includes Escherichia coli KO11.

8. The method of claim 1 wherein the plant fiber material comprises at least about 50% moisture.

9. The method of claim 1 wherein the method further comprises:
slurrying said plant fiber material with water prior to the mixing of b).

10. A method comprising:
mixing a plant fiber material comprising calcium carbonate with at least one lignocellulosic agricultural residue into a mixture such that said mixture is fermentably detoxified for ethanol production by the addition of the plant fiber material alone; and
processing said mixture into ethanol.

11. The method of claim 10 wherein the ethanol yield resulting from processing said mixture is greater than about 50 gallons per ton of the at least one lignocellulosic agricultural residue.

12. The method of claim 10 wherein the at least one lignocellulosic agricultural residue further comprises:
a cotton gin waste
a corn stover;
a rice hull; or
a sugar cane bagasse.

13. The method of claim 10 wherein the plant fiber material comprises a paper sludge.

14. The method of claim 13 wherein the paper sludge comprises:
an ash content ranging from about 5% to about 30% by mass of the sludge, said ash content including the calcium carbonate.

15. The method of claim 3 wherein the paper sludge is selected from:
(a) fine paper sludge;
(b) coated paper sludge;
(c) coated fine paper sludge;
(e) recycle mixed office paper sludge;
(f) recycled newsprint;
(g) de-inked pulp mill sludge;
(h) de-inked paper mill sludge; and
(j) recycled paper sludge.

16. The method of claim 1 wherein the ratio of the at least one lignocellulosic agricultural residue to paper sludge comprises:
from about 1:1 to about 5:1.

17. The method of claim 16 wherein the ratio of the at least one lignocellulosic agricultural residue to paper sludge comprises about 1:1.
